(19) United States
(12) Patent Application Publication
(10) Pub. No.: US 2015/0305662 A1
(43) Pub. Date: Oct. 29, 2015

(54) REMOTE ASSESSMENT OF EMOTIONAL STATUS

(71) Applicant: Future Life, LLC, Washington, DC (US)

(72) Inventors: John S. Conti Kilmer, Washington, DC (US); William D'Alelio, Chevy Chase, MD (US); Jeff Hayes, Elkton, MD (US); Andrew Ramsey, Mount Airy, MD (US)

(21) Appl. No.: 14/625,430

(22) Filed: Feb. 18, 2015

Related U.S. Application Data
(60) Provisional application No. 62/088,777, filed on Dec. 8, 2014.
(60) Provisional application No. 61/985,849, filed on Apr. 29, 2014.

(51) Int. Cl. A61B 5/16; G06K 9/00; A61B 5/00

(52) U.S. Cl. CPC A61B 5/165; A61B 5/164; A61B 5/7275; G06K 9/00315; G06K 9/00281; G06K 9/00261; A61B 2505/07

(57) ABSTRACT

A software product encoding steps for execution by a computer to provide an interactive computer-to-computer link for remote communication between a patient's computer and a therapist's computer, comprising instructions for establishing two-way audio/visual communication between said patient's computer and said therapist's computer, and an emotional recognition algorithm in said patient's computer for recognizing said patient's emotional state.

### FIELD

This invention relates to software and a method for remote assessment of the emotional status of a patient by a psychological or psychiatric therapist.

### BACKGROUND

There is currently a large backlog for providing mental and/or emotional counseling and care to patients, especially for veterans suffering, for example, from post traumatic stress disorder or similar conditions. While this backlog is undoubtedly due to limited staffing and funding for mental healthcare, it is further exacerbated by the centralized nature of healthcare facilities, which is often inconvenient for patients due to their wide geographical dispersion and difficulty in travelling to the healthcare facilities. Additionally, the very nature of mental/emotional healthcare treatments can require frequent visits to the healthcare provider, which results in lack of continuing care for remotely located patients.

Several prior art patent disclosures have endeavored to address one or more of the above-mentioned drawbacks, problems, or limitations of centralized healthcare.

For example, U.S. Published Patent Application No. 2013/0317837 to Ballantyne et al. discloses a method, related system and apparatus implemented by an operative set of processor executable instructions configured for execution by a processor. The method includes the acts of: determining if a monitoring client is connected to a base through a physical connection; establishing a first communications link between the monitoring client and the base through the physical connection; updating, if necessary, the interface program on the monitoring client and the base through the first communications link; establishing a second communications link between the monitoring client and the base using the first communications link; and communicating data from the base to the monitoring client using the second communications link.

U.S. Published Patent Application No. 2011/0106557 to Gazula discloses a framework which allows electronic interactions using real-time audio and video between a patient, family, caregiver, medical professionals, social workers, and other professionals. The framework enables capturing standardized data, records and content of the patients, storing the information captured into integrated Application database and/or into its objects stored in the applications folders and has a screen which provides electronic interaction capabilities using real-time audio and video simultaneous interactions.

U.S. Published Patent Application No. 2012/0293597 to Shipon discloses a method which provides supervision including providing a plurality of information channels for communicating information to the service provider and the user and integrating the information channels to provide access to supervisory functionality for supervising the information channels of the plurality of information channels by way of a single portal. The method provides access to audio/visual functionality, to information record functionality, to diagnostic functionality, to action functionality and to administrative functionality. All functionalities are accessed by way of a portal whereby the portal has access to the functionalities simultaneously. A single accessing of the portal by the user permits the user to gain access to all of the functionalities simultaneously in accordance with the single accessing. The portal can be a web portal. Each of the functionalities is accessed by way of a respective information channel of a plurality of information channels.

U.S. Published Patent Application No. 2013/0060576 to Hamm et al. discloses systems and methods for locating an on-call doctor, specific to a patient's needs, who is readily available for a live confidential patient consultation using a network enabled communication device with a digital camera and microphone. The system facilitates customized matching of patients with doctors to provide higher quality and faster delivery of medical evaluation, diagnosis, and treatment. The systems and methods transmit results through a secure connection and manage a referral process whereby a referring doctor refers a patient to another provider, laboratory, facility, or store for a particular procedure, order, analysis, or care. The referrals may be based on specialties and availability. The system relates particularly to the fields of medicine, where doctors can perform online consultations and provide a diagnosis, treatment recommendations, recommendations for further analysis, triage and/or provide follow up on-call care.

Other prior art patent disclosures have endeavored to provide systems for assessment of emotional states.

U.S. Published Patent Application No. 2004/0210159 to Kilbar discloses a process in which measurements of responses of a subject are performed automatically. The measurements include a sufficient set of measurements to complete a psychological evaluation task or to derive a complete conclusion about a cognitive state, an emotional state, or a socio-emotional state of the subject. The task is performed or the complete conclusion is derived automatically based on the measurements of responses.

U.S. Published Patent Application No. 2007/0066916 to Lemos discloses a system and method for determining human emotion by analyzing a combination of eye properties of a user including, for example, pupil size, blink properties, eye position (or gaze) properties, or other properties. The system and method may be configured to measure the emotional impact of various stimuli presented to users by analyzing, among other data, the eye properties of the users while perceiving the stimuli. Measured eye properties may be used to distinguish between positive emotional responses (e.g., pleasant or “like”), neutral emotional responses, and negative emotional responses (e.g., unpleasant or “dislike”), as well as to determine the intensity of emotional responses.

U.S. Pat. No. 7,857,452 to Martinez-Conde et al. discloses a method and apparatus for identifying the covert foci of attention of a person when viewing an image or series of images. The method includes the steps of presenting the person with an image having a plurality of visual elements, measuring eye movements of the subject with respect to those images, and based upon the measured eye movements triangulating and determining the level of covert attentional interest that the person has in the various visual elements.

U.S. Pat. No. 8,600,100 to Hill discloses a method of assessing an individual through facial muscle activity and expressions which includes receiving a visual recording stored on a computer-readable medium of an individual's non-verbal responses to a stimulus, the non-verbal response comprising facial expressions of the individual. The recording is accessed to automatically detect and record expressional repositioning of each of a plurality of selected facial features by conducting a computerized comparison of the facial position of each selected facial feature through sequential facial images. The contemporaneously detected and recorded expressional repositionings are automatically coded to an action unit, a combination of action units, and/or at least one emotion. The action unit, combination of action units, and/or at least one emotion are analyzed to assess one or more characteristics of the individual to develop a profile of the individual's personality in relation to the objective for which the individual is being assessed.

However, none of the above-recited disclosures is specific to remote systems for mental and/or emotional healthcare.

It would be advantageous if mental and/or emotional evaluations, treatments and counseling sessions could be conducted remotely. Such a system would not only alleviate the necessity of the patient travelling to a centralized healthcare facility, but also enhance the productivity of the healthcare professional by limiting the number of missed or delayed visits by the patient.

### SUMMARY

In one embodiment, the invention resides in a software product encoding steps for execution by a computer to provide an interactive computer-to-computer link for remote communication between a patient's computer and a therapist's computer, comprising instructions for establishing two-way audio/visual communication between said patient's computer and said therapist's computer; and an emotional recognition algorithm in said patient's computer for recognizing said patient's emotional state.

In another embodiment, the invention resides in said emotional recognition algorithm comprising steps for tracking and interpreting changes in digitally-imaged pixel data received by a digital camera connected to said patient's computer over a period of time.

For example, said emotional recognition algorithm can include monitoring changes in shading of pixels imaging said patient's head and/or face by continuously mapping and comparing a topography of the patient's head and/or facial muscles and/or continuously mapping and comparing the patient's eye movements.

Preferably, the emotional recognition algorithm includes steps for tracking changes in pixel data received by a digital camera connected to said patient's computer over a period of time, which changes are correlated with changes in the emotional state of the patient, based upon the patient's facial muscle movements and/or the patient's eye movements.

In another embodiment, the software product further comprises instructions for transmitting signals generated by the emotional recognition algorithm indicating the patient's emotional state over said computer-to-computer link.

Advantageously, the signals from the emotional recognition algorithm installed in said patient's computer are inaccessible to or transparent to the patient, and can include an alarm, alert or other indicator which can be sent to the therapist's computer upon recognition of changes in the patient's emotional state.

Additionally, the software product can further comprise a session recording module in the patient's computer enabling the patient to record the audio/visual session on a computer hard disk in said patient's computer.

In another embodiment, the audio/visual two-way communication is enabled by a digital camera and a microphone connected to said computer and controlled by said software product.

Preferably, the visual two-way communication is enabled by a digital camera having a resolution of at least about 640×480 pixels and a refresh rate of at least about 23 frames/second connected to at least said patient's computer and controlled by said software product.

Additionally, the emotional recognition algorithm can include multiple algorithms for tracking motions of and changes to the patient's facial features including head position, eye position, nose position, skin wrinkling or cheek muscles.

In another embodiment, the software product further comprises a cooperating software product in said therapist's computer enabling reception of remote communications from said patient's computer.

The cooperating software product further comprises an electronic prescription service module installed in said therapist's computer configured to send a prescription order to a prescription provider, and/or an observation recording module in the therapist's computer enabling the therapist to record observations regarding the patient on a computer hard disk in said therapist's computer, and/or a session recording module in the therapist's computer enabling the therapist to record the audio/visual session on a computer hard disk in said therapist's computer.

Another embodiment of the present invention is directed to a method of assessing the emotional state of a patient, comprising establishing two-way audio/visual communication between a patient's computer and a remotely-located therapist's computer; monitoring said patient's visual image with an emotional recognition algorithm provided within a software product installed in said patient's computer; correlating changes in said patient's visual image with emotional states with said emotional recognition algorithm; and transmitting signals indicating said patient's emotional state to said therapist's computer.

Advantageously, according to this embodiment the emotional recognition algorithm comprises steps for tracking and interpreting changes in pixel data received by a digital camera connected to said patient's computer over a period of time.

For example, the changes in pixel data include changes in shading of pixels imaging said patient's head and/or face by continuously mapping and comparing a topography of the patient's head and/or facial muscles and/or continuously mapping and comparing the patient's eye movements.

According to a further embodiment, examples of signals which can be sent include an alarm, alert or other indicator sent to the therapist's computer upon recognition of changes in the patient's emotional state.

In a preferred embodiment, the emotional recognition algorithm comprises tracking motions of and changes to the patient's facial features including head position, eye position, nose position, skin wrinkling or cheek muscles.

### BRIEF DESCRIPTION OF THE DRAWINGS

Further details and the advantages of the applicant's disclosures herein will become clearer in view of the detailed description of [invention], given here solely by way of illustration and with references to the appended figures.

FIG. 1 is an illustration of human facial musculature which may be monitored for changes over time, according to the present invention.

FIG. 2 is an example of a computer program output screen provided to an emotional therapist by the cooperating software product installed in and executed by the therapist's computer.

FIG. 3 is an example of a computer program output screen provided to a patient by the software product of the present invention installed in and executed by the patient's computer.

### DETAILED DESCRIPTION OF THE EMBODIMENTS

Described herein is directed to a software product and its method of use in establishing two-way audio/visual communication between a patient and a remotely-located therapist via a computer-to-computer link between the patient's computer and the therapist's computer. The presently described system provides for enhanced and efficient use of scarce health care resources, by permitting essentially real-time communication between patient and therapist, without requiring that the two be located in the same room.

Each of the following terms written in singular grammatical form: “a,” “an,” and “the,” as used herein, may also refer to, and encompass, a plurality of the stated entity or object, unless otherwise specifically defined or stated herein, or, unless the context clearly dictates otherwise. For example, the phrases “a device,” “an assembly,” “a mechanism,” “a component,” and “an element,” as used herein, may also refer to, and encompass, a plurality of devices, a plurality of assemblies, a plurality of mechanisms, a plurality of components, and a plurality of elements, respectively.

Each of the following terms: “includes,” “including,” “has,” “having,” “comprises,” and “comprising,” and, their linguistic or grammatical variants, derivatives, and/or conjugates, as used herein, means “including, but not limited to.”

Throughout the illustrative description, the examples, and the appended claims, a numerical value of a parameter, feature, object, or dimension, may be stated or described in terms of a numerical range format. It is to be fully understood that the stated numerical range format is provided for illustrating implementation of the forms disclosed herein, and is not to be understood or construed as inflexibly limiting the scope of the forms disclosed herein.

Moreover, for stating or describing a numerical range, the phrase “in a range of between about a first numerical value and about a second numerical value,” is considered equivalent to, and means the same as, the phrase “in a range of from about a first numerical value to about a second numerical value,” and, thus, the two equivalently meaning phrases may be used interchangeably.

It is to be understood that the various forms disclosed herein are not limited in their application to the details of the order or sequence, and number, of steps or procedures, and sub-steps or sub-procedures, of operation or implementation of forms of the method or to the details of type, composition, construction, arrangement, order and number of the system, system sub-units, devices, assemblies, sub-assemblies, mechanisms, structures, components, elements, and configurations, and, peripheral equipment, utilities, accessories, and materials of forms of the system, set forth in the following illustrative description, accompanying drawings, and examples, unless otherwise specifically stated herein. The apparatus, systems and methods disclosed herein can be practiced or implemented according to various other alternative forms and in various other alternative ways.

It is also to be understood that all technical and scientific words, terms, and/or phrases, used herein throughout the present disclosure have either the identical or similar meaning as commonly understood by one of ordinary skill in the art, unless otherwise specifically defined or stated herein. Phraseology, terminology, and, notation, employed herein throughout the present disclosure are for the purpose of description and should not be regarded as limiting.

In the course of a typical therapist/patient counseling session, the therapist, which can be a psychiatrist, a psychologist or other such professional having adequate training in the field, can often detect visual clues from the patient, especially from various facial movements, which enables the therapist to assess the emotional state of the patient. For example, upon asking a question, the therapist often observes the patient's physical responses, such as rapid eye movements, forehead skin wrinkling and the like, which might indicate that the patient is lying or is otherwise negatively affected by the question. Such assessments can provide the therapist with insight as to the patient's condition, which even the patient cannot or will not adequately verbally express.

The primary mode of operation of the present invention is via facial motion amplification (FMA), by which a computer program installed in a computer and connected to a digital camera picks up slight facial motions which allows an emotional counseling therapist to be able to better diagnose a patient who is suffering from PTSD and/or mental illness.

FMA is an imaging algorithm which measures the differences in pixel color and density (such as average contrast change) over time over recognized topological features, to reveal how movement of facial structures change over very small amounts of time; less than a fraction of a second (on order to millisecond events). Topological features are comprised of the musculature of the face, the head, neck, and other body features.

Session data is comprised of capture and storage of real-time audio, video, and processed data associated with algorithms for biofeedback, cross-correlated with FMA data captured in order to achieve emotional reading. Additional algorithms can be applied to measure physiological details of the patient: respiratory, heart rate, blood flow, etc.

Much research and development has been undertaken in the past several decades concerning the detection of emotional changes according to muscle movement in the face. The Facial Action Coding Systems (FACS) was developed in order to characterize facial expressions and in general provide a template structure to communicate these expressions algorithmically.

Paul Ekman and W. V. Friesen developed the original FACS in the 1970s by determining how the contraction of each facial muscle (singly and in combination with other muscles) changes the appearance of the face. They associated the appearance changes with the action of muscles that produced them by studying anatomy, reproducing the appearances, and palpating their faces. Their goal was to create a reliable means for skilled human scorers to determine the category or categories in which to fit each facial behavior. A thorough description of these findings is available only to qualified professionals, by subscription to DataFace, at “face-and-emotion.com/dataface”.

Built upon the FACS, the emotional algorithm of the present invention has been developed to digitally detect facial changes over time and correlate them with emotions from real-time video data. This information is provided to the therapist through computer-to-computer linking of a patient's computer/software product, stored and executed in the patient's computer and a cooperating software product, stored and executed in the therapist's computer.

The software product of the present invention provides live streaming audio/visual service over, for example, an internet connection. This involves essentially real-time capture of video from both the patient and practitioner. A digital camera, such as a webcam, having the capability of accurately interpreting analog visual information from real-life sources and converting this into digital information as a two-dimensional array of pixels over time (video signal), is connected to each computer.

The focus of the video feed is on capturing the faces of patient and practitioner as they are presented to a webcam real-time. The webcam has a perspective of its own which plays into the interpretation of the patient and practitioner subject matter in real-time. As the patient moves relative to the position of the webcam, the software product installed in the patient's computer will have the capability of tracking the head, neck, and upper shoulder regions (when available) of patient in order to more accurately track changes in facial features over time. The software provides live streaming webcam service over an internet connection. The webcam has the capability of accurately interpreting analog visual information from a real-life source and converting this into digital information as a two-dimensional array of pixels over time.

The live streaming webcam service must have a frame rate (or refresh rate) high enough that emotional recognition algorithms (as described below) can accurately sample real-time data and provide consistent results which are trusted and repeatable over a broad range of subject backgrounds (shape of face, disability, and other medical considerations). The digital camera service should have the capability of maximizing the volume of information capture and storage over time for audio, video, and other data and data structures.

The more information which is collected and accurately reproducible, when applied to the emotional recognition algorithms, the more accurate result the algorithms can produce to interpret emotional variations in subject matter (patient or practitioner) over time.

As such, the combined resolution and frame rate of the digital camera system used must be suitable to accurately depict gestures and nonverbal communications for both parties—the patient and psychiatrist/therapist—as if both persons are in the same physical space interacting one-on-one. Obviously, one requirement for accurate visual information retrieval is adequate lighting for the digital camera to record enough information to enable the software algorithms to distinguish subtle differences in facial features over relatively short periods of time.

This involves having a high enough resolution and refresh rate to distinguish changes in facial muscles suitable for topographical construction and deconstruction of regions of two dimensional pixel data. From digital pixel data the algorithm interprets pixel shading such that it can accurately locate the physical objects represented by pixels as the underlying musculature of the face, and how the motions of the face relate to certain nonverbal cues (emotions).

The number of pixels obtained over time is the limiting factor for quality of emotional tracking service. The more information reliably captured by webcam, the more information can be processed for more accurate results as data is processed by real-time algorithms. The combination of real-time tracking of the various skin movements caused by the underlying facial muscle movements that can be associated with emotional response is captured and stored during the live session. Emotional response is cross-correlated, interpreted, and stored as separate data while video is captured. The audio, video, and emotional tracking data are tracked, stored, and can be reviewed at a later time by the therapist.

In one embodiment, the invention resides in a software product encoding steps for execution by a computer to provide an interactive computer-to-computer link for remote communication between a patient's computer and a therapist's computer, comprising instructions for establishing two-way audio/visual communication between said patient's computer and said therapist's computer; and an emotional recognition algorithm in said patient's computer for recognizing said patient's emotional state.

Somewhat counter-intuitively, it is advantageous that the emotional recognition algorithm is present in the software product installed in and executed by the patient's computer, because it is more efficient to process the real-time video data on the client-side (the patient's computer) than the practitioner's computer, since there is relatively less information to be displayed and recorded after processing than before processing.

Additionally, client-side processing ameliorates some limitations of internet signal bandwidth on the accurate recording and representation of emotional cues, which require high frame rate to capture and convey digitally. These events occur on the millisecond scale (fractions of a second), and the patient's native computer operating system is a better platform for the capture and storage of complex and sensitive data relating personal feelings and emotions applied to the complexity of bioinformatics processing requirements.

Thus, the software product in the patient's computer further comprises instructions for transmitting signals generated by the emotional recognition algorithm indicating the patient's emotional state over said computer-to-computer link. Such signals can include but are not limited to an alarm, such as an audio alarm, an alert, such as a visual icon, or any other such indication which can be provided to the therapist's computer upon detection of an important visual clue by the emotional recognition software resident in the patient's computer. These signals can cause the generation of a response, either audibly on a speaker associated with the therapist's computer, or visually on the video screen of the therapist's computer, or both, and require significantly less processing speed and bandwidth than would transmission of a very high resolution image of the patient, sufficient for the therapist to identify an emotional response by the patient. The nature of the emotional responses which can be assessed and sent to the therapist are such as: “patient is lying”, or “patient is angry”, or “patient is distressed”, and the like. Additionally, digitally obtaining and assessing such subtle facial, eye and/or head movements by the emotional recognition algorithm in the patient's software product can help avoid the therapist inadvertently missing such clues during the remote audio/visual session.

In any event, it is advantageous if the visual two-way communication is enabled by a digital camera having a resolution of at least about 640×480 pixels and a refresh rate of at least about 23 frames/second connected to at least said patient's computer and controlled by the software product. Of course, in order to provide audio communication, it is important that a microphone be connected to each computer and controlled by said software products.

The emotional recognition algorithm comprises steps for tracking and interpreting changes in digitally-imaged pixel data received by the digital camera connected to said patient's computer over a period of time. For example, changes in pixel data include changes in shading of pixels imaging said patient's head and/or face by continuously mapping and comparing a topography of the patient's head and/or facial muscles and/or continuously mapping and comparing the patient's eye movements. Rapid eye movement (REM) is identified as one factor in assessing a patient's emotional state, as are variations in the location of the patient's head, and variations in eye position, nose position, skin wrinkling or cheek muscles. Thus, the emotional recognition algorithm includes steps for tracking changes in pixel data received by a digital camera connected to said patient's computer over a period of time, which changes are correlated with changes in the emotional state of the patient, based upon the patient's facial muscle movements and/or the patient's eye movements.

Emotional recognition is accomplished via real-time detection of REM combined with tracking of head, neck, and upper body muscular response and/or position. First, the shoulders, upper body, and neck are tracked if these portions of the body are visible. Shoulders and upper body are not key indicators of emotional response; rather, they are used as a means of tracking movement of the head and face real-time.

For the purposes of tracking the head and face, the algorithm will have the capability of distinguishing between certain physical features. The algorithm will be able to interpret the structure of the face and assign the changing of pixel data over time to these structures as webcam data is processed real-time.

Furthermore, the therapist should be able to accurately determine subtle emotional changes of the face and upper body, as if both parties were actively engaging in the same physical space with limited or no interruption of signal. It may also be advantageous to apply advanced imaging algorithms which can apply “smoothing” or “kerning” effects to the pixels as time progresses.

The data is cross-referenced (correlated) together to interpret emotional states of the patient. Each area of the body tracked will have a visually-recorded representation of their state change over the time for each session. The imaging algorithms have the capability to intelligently correct and enhance images, as well as provide topological data for motion detection. Topological data represents objects which comprise musculature of the face to be interpreted by algorithms as described further.

As the imaging algorithms process data on the client side, it is sent to the therapist connected via a secure channel or portal. In other words, the processed and cross-correlated data is sent from the patient to therapist, and is displayed on the therapist's main screen.

Thus, the software product further comprises instructions for transmitting signals generated by the emotional recognition algorithm indicating the patient's emotional state to the therapist over said computer-to-computer link, and the signals are advantageously inaccessible to or transparent to the patient, such that the patient cannot consciously attempt to avoid such visual clues, important to the evaluation and assessment of his condition by the therapist.

However, it can also be advantageous if the software product installed in the patient's computer has a session recording module enabling the patient to record the audio/visual session on a computer hard disk in said patient's computer, for later review by the patient. Frequently, the patient can forget salient points and advice provided by the therapist during a counseling session. By reviewing a recording of the counseling session, the patient may derive additional benefits from the therapist's statements which may have been missed or not fully understood during the real-time session.

The software product of the present invention can further comprise a cooperating software product in said therapist's computer, enabling reception of remote communications from said patient's computer. The cooperating software product in the therapist's computer can comprise an electronic prescription service module configured with appropriate instructions to send a prescription order to a prescription provider, an observation recording module enabling the therapist to record observations, such as written notes or verbal comments regarding the patient, and a session recording module in the therapist's computer enabling the therapist to record the audio/visual session, each of which can be stored on a computer hard disk in said therapist's computer.

In another embodiment, the present invention is directed to a method of assessing the emotional state of a patient, by establishing two-way audio/visual communication between a patient's computer and a remotely-located therapist's computer, monitoring the patient's visual image with an emotional recognition algorithm, described in detail above, provided within a software product installed in the patient's computer, correlating changes in the patient's visual image with emotional states with the emotional recognition algorithm and transmitting signals indicating the patient's emotional state to the therapist's computer.

As discussed above, the emotional recognition algorithm comprises steps for tracking and interpreting changes in pixel data received by a digital camera connected to said patient's computer over a period of time, such as changes in shading of pixels imaging said patient's head and/or face by continuously mapping and comparing a topography of the patient's head and/or facial muscles and/or continuously mapping and comparing the patient's eye movements. The emotional recognition algorithm includes tracking motions of and changes to the patient's facial features including head position, eye position, nose position, skin wrinkling or cheek muscles.

The signal transmitting step of the method includes transmitting an alarm, alert or other indicator sent to the therapist's computer upon recognition of changes in the patient's emotional state.

Complementing the emotional recognition algorithm is a second algorithm which identifies and optionally records sequences of changes of emotional responses. This second algorithm, termed the sequence algorithm for the present application, is preferably resident only in the therapist's computer. The sequence algorithm identifies and optionally records the changes in the emotional algorithm over time, in response to the therapist's questions to the patient, thus providing the therapist with a real time indication of the changes in the patient's emotional responses during the therapy session which can be recorded and re-evaluated at a later time.

Output from the sequence algorithm represents the linear change in emotional state of the patient over time. Multiple sequences can then be fed-back into the sequence algorithm in order to generate even larger-time-lapse sequences with a generalized emotional state. In other words, if the subject changes from a relaxed to furrowed brow, the emotional recognition algorithm will pick up on the change between relaxed to furrowed, and the sequence algorithm will then ascribe the change in this emotion as a sequence. This sequence is then given an appropriate description such as “anger” or “resentment”.

Sequences are of particular importance because they ascribe human-understandable patterns during a live counseling session. When the therapist asks a specific question and the patient responds, the emotional state can then be validated with greater objectivity by both the emotional recognition algorithm and the sequence algorithm in combination. A marker is placed on the timeline of events when a question is asked by the therapist. During this time, the algorithms are awaiting an emotional change or response by the patient. Once the patient elicits an emotional response, the sequence algorithm will subsequently label the emotional change accordingly.

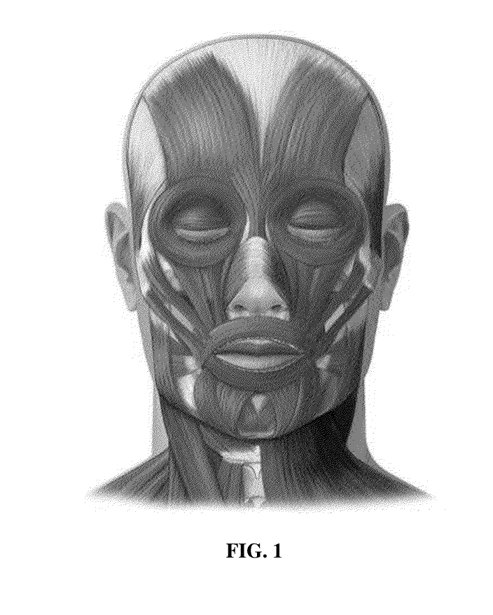

FIG. 1 is an illustration of human facial musculature which may be monitored for changes over time, according to the present invention.

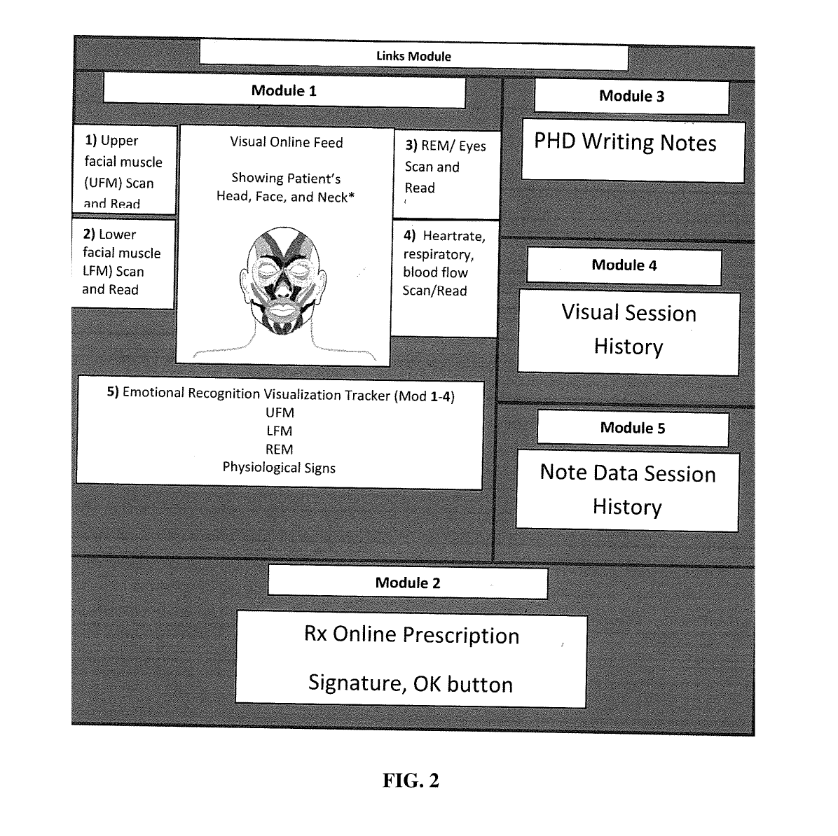

FIG. 2 is an example of a computer program output screen provided to the therapist by the cooperating software product installed in and executed by the therapist's computer. The video area within Module 1 (the “visual online feed”) is viewed as an abstract model of the patient's neck, head, and face. It is not be required that the areas of the body are in view of the webcam; the software product installed and executed in the patient's computer is able to automatically detect and monitor facial muscles separate from the chest and shoulders region which may or may not be in view. Within the model window, it is possible for the algorithm to detect areas of the body from the upper chest and shoulders area up to the top of the head, where particular focus is set on tracking REM and facial muscles for real-time emotional sensing.

Each area of the body within this model window is broken down into separate automated detection algorithms. Each part of the face in question can be monitored real-time with one or several algorithms. The modules can be subdivided into other visual representations of data capture or modular variations of software architecture. The greater the amount of separate information (parts of the body) that is compared at a time, the more accurately the emotional correlation algorithm will interpret changes in emotional state over time.

For example, each of the windows identified as 1) through 4) is a sub-module which provides separate monitoring and analysis of different individual facial responses by the emotional recognition algorithm(s) provided in the patient's computer, which are sent to the therapist. Sub-module 1) can be configured to sense and provide assessment of the upper facial muscles, such as the eyebrows and upper eye facial muscles, which can convey a sense of fear, excitement, anger and the like. Sub-module 2) illustrates scanning of the lower facial muscles, just below the eyes and eye sockets, middle nose and all muscles comprising the mouth, wherein patients express a wide variety of emotions, such as happiness, sadness, jealousy, resent and the like. Sub-module 3) is specific to eye movement tracking, especially REM, and reading of eye direction (pupil vector from source of eye to target relative to webcam perspective). This data can convey that the patient is lying or misleading, as well as providing additional information regarding anger, sadness, happiness and the like. Sub-module 4) can be configured to scan and interpret other indicators of emotional reaction, such as cooling or warming of the patient's face due to changes in blood flow and the like.

The window identified as 5) is another sub-module which provides an overall summary of the various analyses of changes and interpretations from the data provided in windows 1) to 4). Any alarms or alerts which are sent to the therapist can be visually displayed in any or all of windows 1) to 5).

Again in FIG. 2, Module 2 is an online prescription module, by which the therapist can prescribe and transmit to a prescription provider (such as a pharmacy) any medications the therapist deems appropriate for the patient. This function avoids the necessity of the patient visiting the therapist's office to pick up the prescription, and thereby reduces wasted time, material and excessive travel, which will reduce the patient's financial outlay and encourage the patient to obtain the medication in a timely manner.

Module 3 provides the ability for the therapist to take notes relating to the patient during the session. The notes can be written or dictated, and are recorded in the therapist's computer hard drive for later review.

Module 4 provides the therapist with the ability to record the entire session on the hard drive of his computer for later review and analysis.

Module 5 provides the therapist the ability to look back at past session notes. The patient does not have access to Module 5, unless access is granted by the therapist. Certain of these notes can be shared with others by permission of the therapist. Additionally, these past notes can be edited to simplify later searches for them by the therapist. These notes are preferably provided in chronological order.

None of the information provided to the therapist in Modules 1-5 is provided to the patient, and as such is inaccessible to or transparent to the patient.

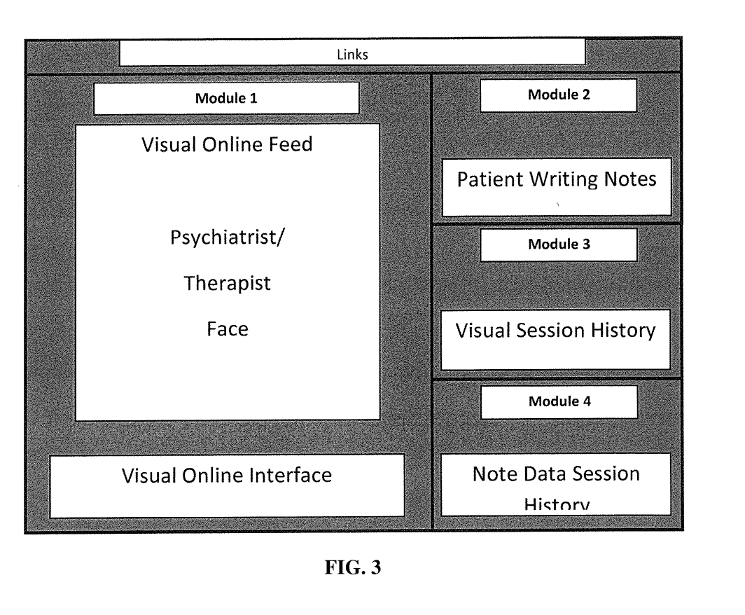

FIG. 3 is an example of a computer program output screen provided to a patient by the software product of the present invention installed in and executed by the patient's computer. Module 1 of the patient's screen is a visual online feed of the therapist's face, provided to enhance the feel of the counseling session to be as similar to an “in-person” or “face-to-face” session as possible. Maximization of the data bandwidth between both users improves accuracy of approximating the analog (“in-person session” event, or “face-to-face”-like behavior) as digital medium through webcam.

Similarly to the therapist's output screen in FIG. 2, Modules 2, 3 and 4 provide the patient with the ability to record his or her own notes, record the visual session and review prior session notes recorded in Module 3, respectively.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A software product encoding steps for execution by a computer to provide an interactive computer-to-computer link for remote communication between a patient's computer and a therapist's computer, comprising:
instructions for establishing two-way audio/visual communication between said patient's computer and said therapist's computer; and
an emotional recognition algorithm in said patient's computer for recognizing said patient's emotional state.

2. The software product of claim 1, wherein said emotional recognition algorithm comprises steps for tracking and interpreting changes in digitally-imaged pixel data received by a digital camera connected to said patient's computer over a period of time.

3. The software product of claim 2, wherein said changes in pixel data include changes in shading of pixels imaging said patient's head and/or face by continuously mapping and comparing a topography of the patient's head and/or facial muscles and/or continuously mapping and comparing the patient's eye movements.

4. The software product of claim 1, wherein said emotional recognition algorithm includes steps for tracking changes in pixel data received by a digital camera connected to said patient's computer over a period of time, which changes are correlated with changes in the emotional state of the patient, based upon the patient's facial muscle movements and/or the patient's eye movements.

5. The software product of claim 1, further comprising instructions for transmitting signals generated by the emotional recognition algorithm indicating the patient's emotional state over said computer-to-computer link.

6. The software product of claim 5, wherein the signals from the emotional recognition algorithm installed in said patient's computer are inaccessible to or transparent to the patient.

7. The software product of claim 1, further comprising a session recording module in the patient's computer enabling the patient to record the audio/visual session on a computer hard disk in said patient's computer.

8. The software product of claim 1, wherein said audio/visual two-way communication is enabled by a digital camera and a microphone connected to said computer and controlled by said software product.

9. The software product of claim 1, wherein said visual two-way communication is enabled by a digital camera having a resolution of at least about 640×480 pixels and a refresh rate of at least about 23 frames/second connected to at least said patient's computer and controlled by said software product.

10. The software product of claim 1, wherein said emotional recognition algorithm includes multiple algorithms for tracking motions of and changes to the patient's facial features including head position, eye position, nose position, skin wrinkling or cheek muscles.

11. The software product of claim 5, wherein said signals include an alarm, alert or other indicator which can be sent to the therapist's computer upon recognition of changes in the patient's emotional state.

12. The software product of claim 1, further comprising a cooperating software product in said therapist's computer enabling reception of remote communications from said patient's computer.

13. The software product of claim 12, further comprising an electronic prescription service module installed in said therapist's computer configured to send a prescription order to a prescription provider.

14. The software product of claim 12, further comprising an observation recording module in the therapist's computer enabling the therapist to record observations regarding the patient on a computer hard disk in said therapist's computer.

15. The software product of claim 12, further comprising a session recording module in the therapist's computer enabling the therapist to record the audio/visual session on a computer hard disk in said therapist's computer.

16. The software product of claim 12, further comprising a sequence algorithm in said therapist's computer configured to identify and optionally record sequences of changes of emotional responses recognized and transmitted by said emotional recognition algorithm in said patient's computer.

17. A method of assessing the emotional state of a patient, comprising:
establishing two-way audio/visual communication between a patient's computer and a remotely-located therapist's computer;
monitoring said patient's visual image with an emotional recognition algorithm provided within a software product installed in said patient's computer;
correlating changes in said patient's visual image with emotional states with said emotional recognition algorithm; and
transmitting signals indicating said patient's emotional state to said therapist's computer.

18. The method of claim 17, wherein said emotional recognition algorithm comprises steps for tracking and interpreting changes in pixel data received by a digital camera connected to said patient's computer over a period of time.

19. The method of claim 18, wherein said changes in pixel data include changes in shading of pixels imaging said patient's head and/or face by continuously mapping and comparing a topography of the patient's head and/or facial muscles and/or continuously mapping and comparing the patient's eye movements.

20. The method of claim 17, wherein said transmitting of signals includes an alarm, alert or other indicator sent to the therapist's computer upon recognition of changes in the patient's emotional state.

21. The method of claim 17, wherein said emotional recognition algorithm comprises tracking motions of and changes to the patient's facial features including head position, eye position, nose position, skin wrinkling or cheek muscles.

22. The method of claim 17, further comprising identifying and optionally recording sequences of changes of emotional responses transmitted to said therapist's computer with a sequence algorithm installed in said therapist's computer.
